(19) United States
(12) Patent Application Publication
(10) Pub. No.: US 2013/0138017 A1
(43) Pub. Date: May 30, 2013

(54) ULTRASOUND GUIDED AUTOMATED WIRELESS DISTRACTION OSTEOGENESIS

(71) Applicants: Jonathon Jundt, Houston, TX (US); Brent Nowak, San Antonio, TX (US)

(72) Inventors: Jonathon Jundt, Houston, TX (US); Brent Nowak, San Antonio, TX (US)

(21) Appl. No.: 13/636,775

(22) PCT Filed: Mar. 24, 2011
(86) PCT No.: PCT/US11/29851

§ 371 Date: Feb. 4, 2013

Related U.S. Application Data
(60) Provisional application No. 61/317,081, filed on Mar. 24, 2010.

(51) Int. Cl. A61N 7/00; A61B 17/66

(52) U.S. Cl. CPC A61N 7/00; A61B 17/66
USPC 601/2; 606/58

(57) ABSTRACT

A bone distraction device applies guided incremental forces to opposing bone segments for the purpose of generating native bone in an osteotomy site (distraction osteogenesis). The bone distraction device automatically adjusts the rate of the distraction utilizing feedback received from the ultrasound transducer and other sensors, using an adaptive decision algorithm(s). A wireless transmitter allows for remote guidance, feedback and monitoring.

### BACKGROUND OF THE INVENTION

1. Field of the Invention

The present invention generally relates to bone regeneration devices. More particularly, the invention relates to an automated ultrasound guided bone distractor that incrementally separates bone segments to induce new bone formation in the distraction site biologically, anatomically and histologically identical to native bone.

2. Description of the Relevant Art

Distraction osteogenesis is a process of inducing natural healing mechanisms in the human body to generate new bone. The rate, quality and time for healing vary from person to person. During distraction osteogenesis, new bone forms under the mechanical condition of gradual incremental traction while simultaneously subject to attenuated functional loads. Distraction osteogenesis begins with the development of a reparative callus between two bone segments separated by a low-energy osteotomy. The osteotomy is an intentional wound akin to a fracture that results in the recruitment of osteoprogenitor cells, followed by osteoinduction then osteoconduction through the establishment of a biological template. There are five stages associated with distraction osteogenesis. First, the osteotomy is performed. Second, a latency period is allowed for the callus to form in the osteotomy site. Third, the law of tension stress is applied via a device using incremental movement of the proximal and distal bone segments away from each other. During this active distraction phase, tension applied to the tissues results in a prolongation of angiogenesis with subsequent increased tissue oxygenation and increased fibroblast proliferation and biosynthetic intensification. Fourth, active distraction ceases and a period of consolidation ensues. This stage allows the healing process to produce an adequate amount of bone at the desired extension length. The fifth stage of distraction osteogenesis is a reorganization of the bony tissue known as remodeling. The remodeling stage enhances the integrity of the regenerate.

Currently, several factors result in suboptimal distraction osteogenesis devices. First, there exist no biologically based distraction osteogenesis devices. That is, current devices whether automated or not are incrementally expanded at a determined rate irrespective of the patients' physiologic and biologic response to therapy. A need exists in the art for a biologically based implantable or external distractor that actively stimulates healing through ultrasound or related electromagnetic stimulation; continuously measures the biological and structural state of the distraction site; and adapts the distraction rate. A new distractor must be easy to apply, aesthetic, safe, should reduce morbidities associated with the current art including scarring and infection, and should eliminate the potential for morbidity due to patient noncompliance. Second, there exists no real-time, in-situ measurement system (e.g., sensor) that is needed to record the current state of healing. A need exists to capture the current state of the complex, coupled physiologic and biologic response to distraction therapy. Third, mathematical representations (control laws) do not exist that map the sensor signal to the respective healing stage and degree of healing in that stage as it relates to the existing native bone. A need exists to utilize the highly non-linear, coupled sensor response in a control system that accommodates a broad variety of unique biologic parameters in an adaptive fashion in order to determine the appropriate distraction rate, in an automated distraction device.

### SUMMARY OF THE INVENTION

In one embodiment, a bone distractor that overcomes at least some of the problems noted in the prior art includes a first member couplable to a first portion of a bone to be distracted during use and a second member couplable to a second portion of a bone to be distracted at a position spaced across a separation in the bone from the attachment site of the first member during use. The bone distractor also includes an actuator coupling the first member and the second member, wherein the actuator moves the second member relative to the first member during use. A sensor unit is positioned to send sensing signals to the callus between the first member and the second member during use. During use a controller of the distractor, coupled to the actuator and the sensor unit, determines biologic characteristics of the callus based on readings obtained from the sensor unit during use, and operates the actuator in response to the determined biologic characteristics of the callus during use. In an embodiment, the sensor unit is an ultrasound unit configured to send ultrasound signals to the callus between the first member and the second member. The distractor may be external to the bone or may be implantable. In an embodiment, the distractor includes a transmitter/receiver capable of transmitting and/or receiving control signals during use. In an alternate embodiment, the distractor includes a second ultrasound therapy system, in conjunction with or as a separate element of the ultrasound measurement (sensor) system. The ultrasound therapy system stimulates healing.

A distractor may be used to distract a bone. In an embodiment, a first member of a distractor is coupled to a first portion of the bone. A second member of the distractor is coupled to a second portion of the bone at a position spaced across a separation in the bone from the attachment site of the first member. Each bone segment is subjected to sensing prior to engaging the first and second portions to gain a baseline bone density unique to each patient. This baseline scan may be performed prior to insertion of the distractor or after the bone distractor is placed into the patient (using the sensor unit of the bone distractor), prior to initiation of the distraction program. Operation of the distractor is initiated and the first and second members are incrementally separated from each other. After a predetermined amount of time has passed a sensor unit positioned in, or proximate to, the distractor, is operated to send sensing signals to the bone. The sensing signals may be used to determine the physical characteristic (density) of the callus. The further movement of the first and second member away from each other is controlled, in part, by the determined biological characteristics of the callus. Callus measurements and distraction of the members, based on the determined physical characteristics of the callus, is continued until the appropriate distraction is achieved. In an embodiment, the sensing unit is an ultrasound unit.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 depicts an embodiment of a bone distractor;

FIG. 2 depicts an embodiment of a bone distractor coupled to a bone;

FIGS. 3A and 3B depict schematic diagrams of the positioning of acoustic sensing elements in a longitudinal transmission orientation;

FIGS. 4A and 4B depict schematic diagrams of the positioning of acoustic sensing elements in a through-transmission orientation;

FIG. 5 depicts a schematic diagram of a bone distraction system;

FIG. 6 depicts a schematic diagram of a controller; and

FIG. 7 depicts a schematic diagram of an adaptive decision element.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

It is to be understood the present invention is not limited to particular devices or biological systems, which may, of course, vary. It is also to be understood that the terminology used herein is for the purpose of describing particular embodiments only, and is not intended to be limiting. As used in this specification and the appended claims, the singular fauns “a”, “an”, and “the” include singular and plural referents unless the content clearly dictates otherwise.

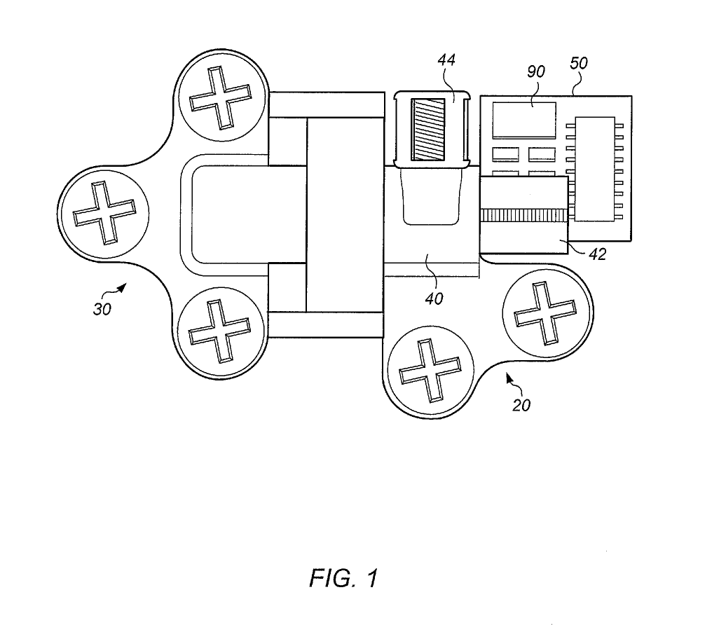

FIG. 1 depicts an example of a bone distractor 10 that may be used for any bone or bone portions in the body. Bone distractor 10 includes a first member 20 which is couplable to a first portion of a bone to be distracted. A second member 30 is couplable to a second bone portion opposing the first bone portion. An actuator 40 couples first member 20 to second member 30. As used herein the term “actuator” refers to any device for moving or controlling a mechanism or system. An actuator is a device that takes energy, usually transported by air, electric current, or liquid, and converts the energy into some kind of motion. For example, an actuator may create kinetic forces that move the coupled portions of the body (e.g., vessels, limbs, jaws, etc.).

In one embodiment, actuator 40 moves first member 20 and second member 30 apart from each other in response to a control signal (e.g., an electrical signal). Examples of actuator 40 include, but are not limited to, electric motors, shape-memory effect driven actuators, or pneumatically or hydraulically operated piston actuators. In one embodiment, actuator 40 includes an actuation component 42 and a drive component 44. Drive component 44 couples moves actuation component 42 in either direction. In an embodiment, actuation component 42 may be a threaded member (e.g., a threaded bolt, rack-and-pinion, cam mechanism, linkages, or the like), which is turned or otherwise motivated by drive component 44 during use. In embodiments where actuation component 42 is a threaded member, the drive component 44 interacts with the threaded portion of actuation component 42 to drive the first member and second member in opposite directions. First member 20 and second member 30 are coupled to actuator 40 such that the first member and the second member moved away from each other during distraction. Actuator 40, in some embodiments, may be operated to move first member 20 and second member 30 toward each other.

Generally, actuator 40 is configured to move the bone portions away from each other, increasing the gap between the bone portions so that new bone material grows in the newly formed gap. This “longitudinal” movement is the most common form of distraction. In some embodiments, actuator 40 may be designed to allow alternate movement of the bone positions. For example, actuator 40 may be designed to allow movement of bone portions lateral to each other (e.g., perpendicular to the longitudinal movement). Actuator 40 may be configured to allow any movement along any arbitrary X-Y-Z axis. Furthermore, actuator 40 may be configured to rotate the bone or bone portions with respect to each other.

Actuator 40 may be controlled using a controller 50 disposed in, or proximate to, distractor 10. Controller 50 may be configured to provide electrical signals to actuator 40 to operate the actuator 40 during use. Distractor may also include an internal power supply for supplying power to controller 50 and actuator 40. Controller 50 may include one or more communication units 90 to transmit data collected by the distraction system sensors during use. A communication unit 90 may be a transmitter/receiver. Data obtained from ultrasound measurements of the bone may be stored in controller 50. Such information may be useful to a practitioner for monitoring the progress of the distraction. Data stored in controller 50 may be received by a practitioner using transmitter/receiver 90 when an appropriate wireless signal is received by the transmitter/receiver (e.g., via a Bluetooth communication system). In an embodiment, a practitioner may send a signal to controller 50 through transmitter/receiver 90 to obtain collected data. Upon receipt of the request for data, controller 50 may send a signal to transmitter/receiver 90 to send the requested data to a reading device of the practitioner. The practitioner may review the device feedback and may modify the current distraction distance and/or rate in response to the analysis, by sending a signal to the controller to modify the rate or distance of the distraction. In this manner the practitioner may monitor and control the distraction process.

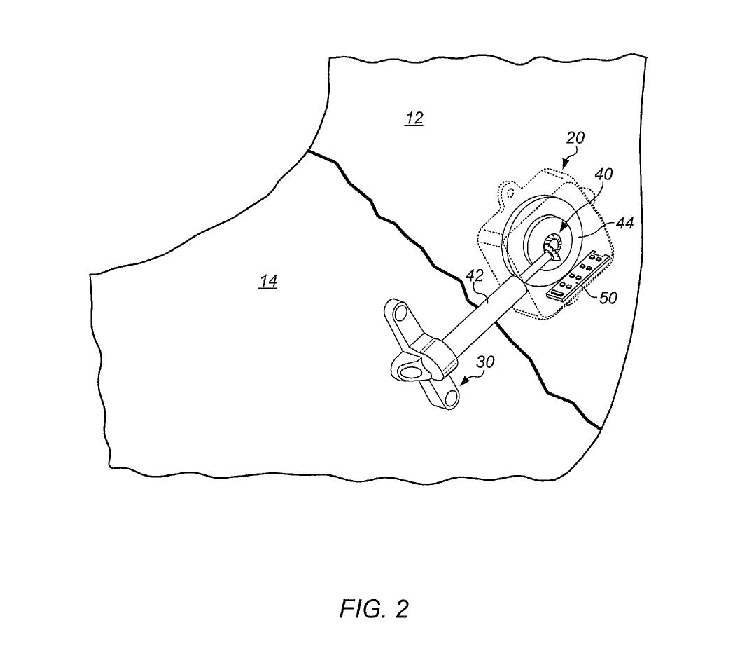

FIG. 2 depicts an embodiment of a bone distractor device coupled to a bone. A first member 20 is coupled to a first portion of 12 of a bone. A second member 30 is coupled to a second portion 14 of a bone proximate to the first portion. An actuator 40 couples first member 20 to second member 30. Actuator 40 includes an actuation component 42 and a drive component 44. Actuator 40 may be controlled using a controller 50 disposed in first member 20 of the distractor system.

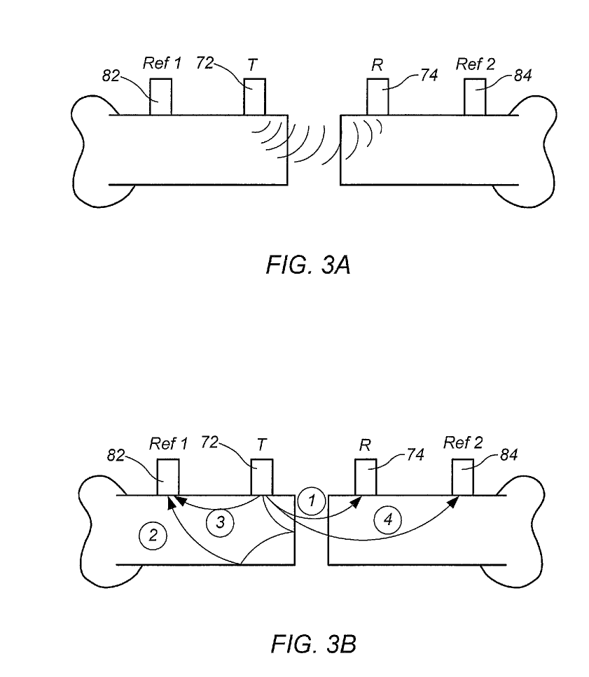

Distractor 10 includes sensor units, which are capable of determining the physical characteristics of a bone coupled to first member 20 and second member 30. Examples of sensor units that may be used include, but are not limited to, acoustic measurement units. A first acoustic measurement configuration, as shown in FIG. 3A, is defined here as longitudinal transmission. In longitudinal transmission one acoustic measurement unit is the transmitter 72. Transmitter 72 is mounted to the bone, subcutaneously on one side of the osteotomy. The second acoustic measurement element is the receiver 74. Receiver 74 is mounted to the bone, subcutaneously on the opposite (or second) side of the osteotomy. The first and second elements may also be referred to as transceivers. That is, the acoustic elements may be configured/commanded as both transmitters and receivers, which are operationally monitored and controlled by controller 50. These acoustic measurement elements, as used herein, therefore imply all operational modes. In addition to the acoustic measurement elements, reference elements 82 and 84 may also be used. Reference elements (82, 84), may be acoustic measurement sensors that operate primarily as receivers. In one embodiment, reference elements are mounted on either side of the osteotomy. The reference elements measure direct longitudinal acoustic signatures, as well as echo pulses that reflect off of acoustic impedance mismatches that occur at the osteotomy site. FIG. 3B depicts several possible acoustic paths between the transmitter 72 and receiver 74 and reference elements 82 and 84. When the acoustic measurement units are operated as a transmitter, they may vary pulse width, frequency content, and duration. The received signal strength(s) and time delay(s) by each acoustic measurement unit and/or reference unit is pre- and post-processed and are inputs to the controller. Subsequently, the controller may redefine the acoustic transmissions, acoustic-healing, or distraction modes.

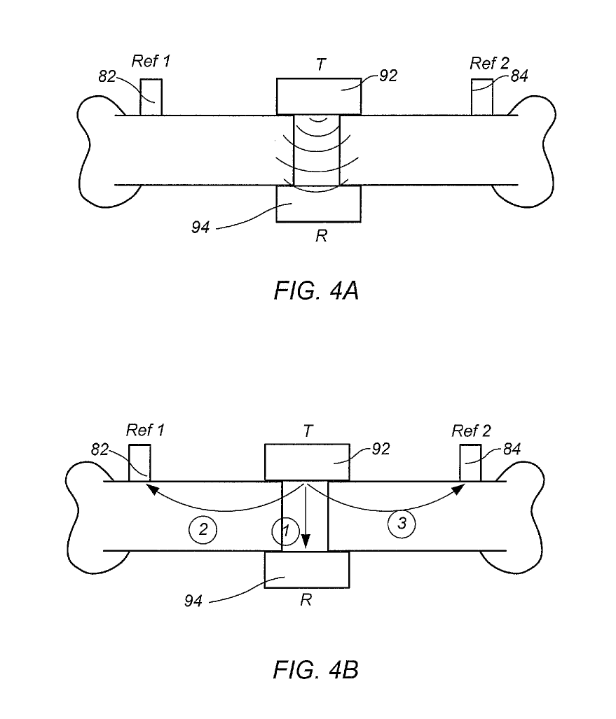

A second acoustic configuration, as shown in FIG. 4A, is defined here as through-transmission. In the through transmission configuration one acoustic sensing element is the transmitter 92. Transmitter 92 is mounted to the bone, subcutaneously on one side of the osteotomy. Second acoustic element is the receiver 94. Receiver 94 is mounted to the bone, subcutaneously on the opposite (or second) side of the osteotomy. The first and second elements may also be referred to as transceivers. That is, the acoustic elements may be operated as both transmitters and receivers, which are operationally monitored and controlled by controller 50. These acoustic measurement elements, as used herein, therefore imply all operational modes. In addition to the acoustic measurement elements the system employs acoustic reference elements 82 and 84. Reference elements (82, 84), are acoustic sensors that operate primarily as receivers. One reference element is mounted on either side of the osteotomy. The reference elements measure direct longitudinal acoustic signatures, as well as echo pulses that reflect off-of acoustic impedance mismatches that occur at the osteotomy site. FIG. 4B depicts several possible acoustic paths between the transmitter 92 and receiver 94 and reference elements 82 and 84. When the acoustic measurement units are operated as a transmitter, they may vary pulse width, frequency content, and duration. The received signal strength(s) and time delay(s) by each acoustic measurement unit and/or reference unit is pre- and post-processed and are inputs to the controller. Subsequently, the controller may redefine the acoustic transmissions, acoustic-healing, or distraction modes.

In some embodiments, acoustic elements are ultrasonic transmitters and receivers. Ultrasonic transmitters and receivers operate at frequencies of 200 kHz to 1.5 MHz, with ultrasound pulsed at about 1 kHz, about 20% duty cycle, and up to about 30 mW/cm² intensity. Ultrasound units are programmable such that the exact frequency used for a given person or animal may be adapted to the patient's or animal's biological characteristics. For example, the use of ultrasonic monitoring allows of the ability of the device to respond/detect local bone environment variations in the population. Since no single osteotomy will be identical, an advantage of an ultrasound based distractor is that it can automatically adjust for the particular quality/quantity of bone for each patient.

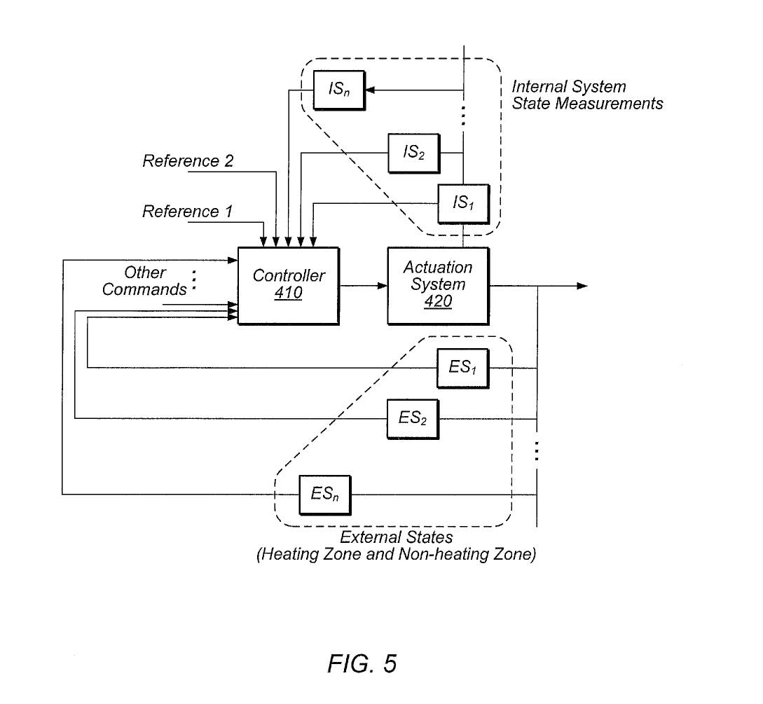

A bone distractor, as described in any of the embodiments described herein, is a platform that may be utilized on any bone of the human (or other) skeletal system. A schematic diagram of a bone distraction system, as shown in FIG. 5. The bone distraction system includes a controller 410, an actuation system 420, sensor units, acoustic sensing elements, and communication units. The positioning of the acoustic units is depicted FIGS. 3 and 4. The sensor units are further defined by operational and functional characteristics as the internal-state sensor units (IS₁, IS₂, . . . ISₙ) and the external-state sensor units (ES₁, ES₂, . . . ESₙ). The acoustic sensing elements are further defined by operational and functional characteristics as acoustic-measurement elements and acoustic-healing elements. As discussed with regard to FIGS. 3 and 4, one or more reference elements (R₁, R₂, . . . Rₙ) are coupled to controller 410.

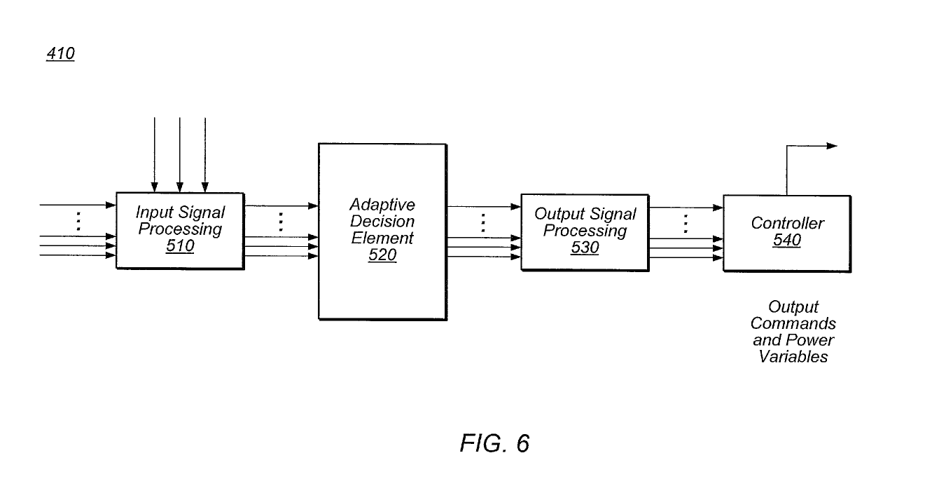

The distraction system provides unexpected benefits beyond acoustic sensing or other sensing or incremental distraction osteogenesis alone or in combination. Controller 410, shown in FIG. 6, includes an input signal processing element 510, an adaptive decision element 520, an output signal processing element 530, and a controller element 540. This represents a multiple-input, multiple-output (MIMO) system. The density of real-time data, processing, and decision making enables controller 410 to characterize and adapt to individual patients and their unique healing conditions.

Each of these controller elements may be implemented in either software or hardware or in some combination of software and hardware. The hardware may include, but is not limited to, a microprocessor (CPU, GPU, FPGA, dedicated Fuzzy Logic chips), memory (RAM, Flash Memory, optical memory), A/D converter, D/A converter, bandwidth filters, amplifiers, discrete circuits, analog circuits, or other electronic components at various implementation levels. The software implementation levels include, but are not limited to, code at the application level, the decision-making level, the operational level, safety/fail-safe level, driver level, or in machine code or some other instantiations of software. The hardware implementation levels include, but are not limited to, individual electronic components, as those listed above and others, and/or integrated circuits of the type VLSI, ULSI, System-on-a-chip, or even a three-dimensional integrated circuit and others to be conceived. Functionally, the input and output signal processing units include, but are not limited to, signal acquisition, signal amplifiers, filters, or pre-conditioning software and hardware.

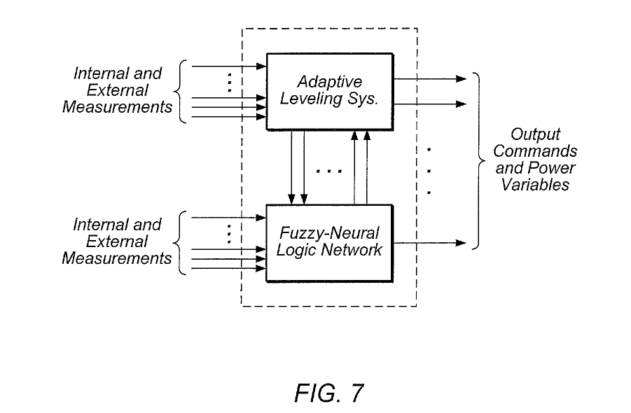

The sensor units monitor internal system states and external states, which are then defined as internal-state sensor units, (IS₁, IS₂, . . . ISₙ), and external-state sensor units, (ES₁, ES₂, . . . ESₙ). Internal-states refer to and measure the distraction mechanism and its operation that may include, but are not limited to, linear and/or rotational kinetics (actuator force, torque, pressure), strain, strain rate, rotational and/or linear kinematics (position, velocity, acceleration), acoustic pulse transmit enable/disable, acoustic pulse receive enable/disable, operational conditions (current, battery power), and temporal conditions (time, counters, delta-time, clocks), etc. In the above description, these internal-states refer to a specific actuation energy domain. Specifically, the internal states listed refer to the electro-mechanical energy domain. As described elsewhere, the distraction mechanism may employ other individual energy domains as a motive source or combinations thereof; such as but not limited to, electro-hydraulic, electro-magnetic, electro-magnetic-mechanical, electro-pneumatic-mechanical, magneto-resistive, piezoelectric, etc. The description of the sensing modes necessary to monitor internal states and control other motive sources can be described with respect to the energy domain(s), as needed. Internal and external state information is communicated to the adaptive decision element, as depicted in FIG. 7.

External-states refer to and measure modes of the biological environment that may or may not be in the healing zone. Within healing zone, healing occurs through overlapping phases. The external-state sensors combine to determine the healing phase, which includes determining the magnitude, rate, and volume/density of hematoma, soft callus, collagen bundles, osteoid deposition and mineralization, microcolumn formation and final ossification stages. The sensing modes may include, but are not limited to, the temperature, pH, oxygen saturation, etc. Along with information regarding the physical information of the callus, the adaptive decision element may use the information obtained from the external state sensors to determine the progress of the healing, and adjust the distraction rate accordingly.

The communication unit provides wireless Bluetooth communication between the controller and a receiving device (computational platform) within near proximity of the healing zone. The communication unit allows the surgeon to monitor the healing process without the use of x-rays or invasive means.

Distractor systems may be implanted into the patient at the osteotomy site, or may be positioned externally on the patient, proximate to the osteotomy site. During use, distractor system is coupled to the bones, or bone portions, using one or more fasteners to couple each portion of the distractor to the bone or bone portions. In an embodiment, the fasteners are screws that are formed from a biocompatible material. In an embodiment, fasteners may be formed from an inert material (e.g., titanium) or may be formed from a biodegradable material.

For proper distraction rates to be determined, it is desirable to have a “baseline” scan of the bone or portion of the bone to be distracted. The baseline scan allows factors such as the current bone density and quantity to be taken into account before a distraction rate is determined. In an embodiment, a baseline scan may be performed prior to coupling distractor 10 to a bone or portions of a bone. In other embodiments, a baseline scan may be performed after distractor 10 is coupled to the bone, using the ultrasound transmitter/receiver to determine the bone characteristics. The baseline scan does not affect the initial rate of distraction, rather it provides a patient specific distraction “goal” for density rather than an idealized density. For example, the initial distraction rate may begin continuously at day 0 at about 0.25 mm/day during what was once considered the latency phase. Then, based on device feedback, it will increase or decrease the speed of distraction based on site specific feedback. In some embodiments, baseline scan information may be collected using x-ray (e.g., CT) or magnetic imaging (e.g., MRI). Information collected using x-ray or magnetic imaging techniques may be provided to the controller of the bone distraction device and may be used to plan the distraction goals. Information obtained form imaging techniques may be used in place of or in combination with baseline scan information obtained using the distractor system.

During use, broad band electrical pulses of ultrasonic frequencies are sent from ultrasound transmitter 72 to the ultrasound receiver 74 along a transmission axis. Ultrasound transmitter 72 and ultrasound receiver 74 are positioned such that the transmission axis passes through newly forming bone material 80 between first bone portion 12 and second bone portion 14. The receiver receives the acoustic signals directed along the transmission axis and relays them to controller 50 which analyzes the received acoustic signal as distorted by the imposition of the bone material between ultrasound transmitter 72 and ultrasound receiver 74. The controller may determine a bone density of the bone matter 80 disposed between the bone portions. Details regarding the measurement of bone density using ultrasound may be found in U.S. Pat. No. 6,364,837 to Mazess et al., which is incorporated herein by reference.

Once physical characteristics of the new bone material (e.g., bone density) has been determined, controller 50 may send control signals to actuator 40 to allow the two portions of the bone to be separated at a rate appropriate for the healing process of the patient. For example, the rate of separation may be increased if the new bone material appears to be sufficiently dense at the current distraction rate. Alternatively, the rate of separation may be reduced if the new bone material appears to have a low density. Controller 50 may be programmed to perform the necessary calculations to determine the appropriate distraction rate based on the measured bone density unique to each patient.

Additionally, controller 50 may be programmed to take into account patient specific factors such as gender, nutritional status, hematological profile, medication, activity level, age or genetics. One or more of these factors may be incorporated into an algorithm that controls the rate of distraction. The controller may use algorithms based on artificial intelligence, including heuristic and non-heuristic methods. Specific types of artificial intelligence include, but are not limited to, fuzzy logic systems, neural networks, fuzzy neural networks, genetic algorithms, sliding mode control, adaptive algorithms, and other heuristic, non-deterministic, or deterministic methodology, such as but not limited to, Bayesian, probabilistic, expert systems, or optimal control methods. The artificial intelligence may employ any combination of the previously listed control modes. These algorithms may be incorporated into the adaptive decision element, as shown in FIG. 7.

In an embodiment, controller 50 is configured to periodically determine the physical characteristic of the bone matter disposed between the first bone portion and the second bone portion. For example, controller 50 may be configured to operate the ultrasound unit to determine the bone density in periods such as continuous, at least every 20 minute, at least every hour, at least every two hours, at least every four hours, at least every six hours, or at least every 10 hours. The difference in bone density between each test may be used to determine the current rate of growth for new bone material between the first and second bone portions. Growth rate, along with the determined bone density, may be used to determine the appropriate distraction rate at any given time. This method will allow adaptive and learning modalities to be incorporated into a distraction device.

In an embodiment, distractor operation is described in three general modes: Installation-Calibration, Normal Operation, and Termination/Fail-Safe. All modes are described in this section from the perspective that the initial surgical protocols/procedures have been conducted to provide the surgeon access to the osteotomy site independent of cause, i.e. fracture or scheduled distraction osteogenesis.

The Installation-Calibration operation is used for initial set-up of the device. This description applies to either acoustic-measurement configuration (longitudinal or through-transmission). The primary function of this first process is to characterize the bone with the installed sensors at the osteotomy site. For the planned distraction osteotogenesis procedure the transmission-receiver calibration will first be conducted prior to creating the osteotomy. This calibration procedure will then be repeated after the osteotomy site is created. This calibration procedure will run through a series of frequencies, pulse widths, durations, and other variations of transmission and reception to confirm that all systems are operational. After functionality is confirmed, the site will be characterized acoustically. Patient characteristics (age, gender, surgical site (e.g. femur vs. mandible), etc) may be entered.

When applied to a fracture site the calibration procedure may employ a longitudinal transmission configuration on either side of the fracture site. This calibration procedure will run through another series of frequencies, pulse widths, durations, and other variations of transmission and reception to confirm that all systems are operational. After functionality is confirmed, the site will be characterized acoustically. Patient characteristics (age, gender, bone, etc.) may be entered.

In one embodiment, a data set, referred to here as the DO/Fracture Healing Data is created. The DO/Fracture Healing Data provides a priori data that is used in the adaptive decision element 520, which may include, but is not limited to, Bayesian data fusion, Partial Response Maximum Likelihood, and other decision methods. The DO/Fracture Healing Data allows new healing protocols to be developed for the complete patient population. Conceptually, this will allow general patient characteristics, (e.g. age, gender, height, weight, etc.), as well as atypical or non-optimal patient characteristics, (e.g. smokers, diabetics, etc.), to be gathered and incorporated in the adaptive decision element.

The Normal Operation is used after the initialization-calibration operation is completed. The Normal Operation varies throughout the stages of healing. Initially a latency period is prescribed by existing protocols. This latency period is between 7 and 14 days depending upon the bone, patient, and other criteria. These criteria have been entered into the device during the Installation-Calibration procedure. During the latency period the distraction actuation is disabled. The Internal-State sensors monitor the health of the system to assure that Normal Operation-Idle Mode is maintained. If battery levels or currents are measured at the actuator site, then the system will transition to a Termination/Fail-Safe mode. During the latency period in the idle mode, the external-state sensors will monitor the health of the healing zone. That is, infection may be indicated by a local rise in temperature, changes in pH, acoustic properties, etc. This provides the ability to introduce antibiotics pro re nata, rather than generally prescribed for all procedures. Also, it allows the physician immediate information prior to the general degradation of the patients health, thereby, minimizing complications, device rejections, and/or additional surgical procedures. The external-state sensors (FIG. 5) and adaptive decision element (FIG. 6, 520) continuously provide this monitoring capability.

For the Distraction Osteogenesis application, the Normal Operation enters into the Distraction Mode. The Distraction Mode provides continuous distraction and monitoring subcutaneously. For the Fracture application, the Normal Operation enters into the Monitor Mode. The Distraction Mode may be disabled or the distraction mechanism may not be installed. Continuous loading provides improved healing conditions and rates. In addition, the loading may be modified to provide compressive and tensile loading cycles that may be designed for each patient, the bone site, and the unique healing characteristics that are encountered real-time. Each healing stage may have a unique decision making process, as needed and shown by clinical data, such that the algorithms may be defined as Normal Operation-Distraction Mode-Stage 2, Normal Operation-Distraction Mode-Stage 3, etc. During these stages, the physician will monitor the progress through the communication unit. The data may be uploaded to the base unit, studied by the physician, and operational parameters may be modified, if needed. This interface allows the physician to correlate patient feedback (pain, discomfort, or other conditions reported) to the operational mode, and modify if needed.

The classic distraction therapy prescribes a discrete incremental change in distraction distance (gap) of 1.0 mm/day. This classic distraction therapy is patient directed, non-continuous and, typically is increased four times per day at 0.25 mm per activation. This therapy is applied to the total patient population regardless of osteotomy site, patient age, gender, compliance history, etc. However, the healing process is quite complex (inflammation, soft callus formation, hard callus formation, patient age, comorbidities). While cellular mechanisms are largely understood, it is the communication between cells to secrete, multiply, adhere or detach and move that are not entirely understood. It is, therefore, self-evident that healing will benefit by sensing and controlling in an intelligent, adaptive means.

Use of ultrasound monitoring of the physical characteristics of newly forming bone matter has the ability to allow changes to the distraction profile from a discrete incremental step function to the most appropriate distraction profile for an individual. One alternate distraction profile that a device having ultrasound monitoring provides is a continuous distraction at a linear rate over a 24-hour period for the same total distraction of 1.0 mm or greater depending on sensor feedback. Another alternate distraction profile that our device can employ is an “S” curve profile.

The Termination/Fail-Safe Mode is self-descriptive. The mode is enabled when any abnormal conditions are sensed. Excessive current when the actuator is not commanded will enable Termination/Fail-Safe. Other examples include excessive temperature change of the device (prevent subcutaneous burns); loss of sensors (pH, temperature, etc.); excessive distraction or contraction rates; and others. The mode may set an audible alarm, enabling the patient to contact the physician or to engage a manual shutdown (these modes are typically found in subcutaneous devices today such as pace-makers). The Termination/Fail-Safe mode may be entered under normal conditions, such as, the completion of the distraction length or duration. A schematic diagram of adaptive decision element 520, is shown in FIG. 7. In one embodiment, adaptive decision element 520 includes an adaptive learning system and a fuzzy-neural heuristic algorithm. Not shown in FIG. 6 is the storage of the DO-Fracture Healing Data set. This data may be used in a variety of fashions, including but not limited to, to define soft limits, hard limits, a priori probability density functions, look-up tables, and used in matching waveforms for Probable Response Maximum Likelihood (PRML) signature analysis or the like.

Adaptive decision element 520 is a multi-threaded, real-time process. The primary level (highest priority thread) of operation for the adaptive decision element is Health Monitoring. Health Monitoring applies to both the Internal State (i.e. the device) and External State (i.e. the healing zone). The Health Monitoring will be non-interruptible in a multi-thread real-time deterministic operating system. That is, at any stage health degradation will over-ride all other operational modes and immediately transition to the Termination/Fail-Safe mode.

A second thread will monitor the Healing Stage. The Healing Stage is unique and differs from the Health Monitoring. The Healing Stage thread provides continuous monitoring of the healing process to determine which stage in the normal healing process the osteotomy site exists. The fundamental sensing mode is acoustic. The acoustic response as characterized by magnitude, the time delay between transmit and receive, and the waveform signature. Since the communication medium is changing in real-time, continuous monitoring is necessitated in continuous distraction modes. The Adaptive Decision Element may identify unique acoustic response characteristics in Stage 1 that fall within normal parameters, yet may allow increased distraction rates in Stage 2 other modified tensile/compression sequences, for example.

In some embodiments, multiple distraction devices may be substantially simultaneously implemented to perform a reconstructive bone procedure. Cases of hemifacial microsomia, hemimandibular hypoplasia, cancer, trauma or other unilateral conditions are more common than bilateral indications. However, in some installations, bilateral distraction devices may be indicated. Bilateral applications are common in limb lengthening procedures. These procedures increase an individual's vertical height through bilateral distraction osteogenesis of the leg limbs. An asymmetric distraction activation sequence would result in unequal leg length and subsequent gait disturbances. For these cases, another thread will monitor and synchronize the distraction process. In an embodiment, the transmitter/receiver of one or more of the distractors may be used to transmit/receive information regarding the progress of distraction at other sites. One or more of the distractor controllers may include algorithms to take into account the information provided by multiple distractors and adjust the distraction rate accordingly. For example, a distraction rate may be decreased at one or more distractors if the progress of the distraction at those sites is proceeding faster than the distraction at other sites. In this way, differences in bone quality/quantity at different sites of a patient may also be taken into account to ensure that a synchronized distraction is obtained.

Distractor systems described herein may also be used for non-distraction applications. For example, a distractor system may be used to heal bone fractures. When used in such applications, the distractor system may simply hold the bone portions in place without moving the bone portions away from each other. The sensor unit, however, may be activated to monitor and speed the progress of healing.

In one embodiment, a distractor sensor system may be used to treat and monitor bone density in patients with osteoporosis or osteopenia. Use of the sensor guided distractor systems may be used to monitor changes in bone density in affected areas.

An ultrasound unit, controller and transmitter/receiver may be incorporated into any type of distractor system. For example, an ultrasound unit, controller and transmitter/receiver may be incorporated into an actuator powered by intermittent electrical current flow through a shape-memory-effect (SME) actuation component. The controller would provide the appropriate electrical current to the shape-memory effect wires to operate the actuation component. Examples of a distraction device that uses shape-memory effect actuator are described in U.S. Pat. No. 6,033,412 to Losken et al., which is incorporated herein by reference.

In another embodiment, sensor units may be used in a non-automated distraction device. For example, with reference to FIG. 1, a distraction device may include a first member 20 which may be coupled to a first portion of a bone. A second member 30 which may be coupled to a second portion of a bone proximate to the first portion. An non-automated actuator 40 couples first member 20 to second member 30. Actuator 40 includes an actuation component 42 and a drive component 44. In a non-automated distraction system, the drive component of the actuator is manually activated. Controller 50 may be used to monitor the progress of the distraction, collect information regarding the progress of the distraction and the state of the distractor system, and transmit the information to the user of the distractor system.

In another embodiment, an ultrasound unit, controller and transmitter/receiver may be incorporated into an actuator powered by a small electric motor. The controller would provide the appropriate electrical current to the motor to operate the actuation component. Examples of a distraction device that use an electric motor actuator is described in U.S. Pat. No. 5,976,138 to Baumgart et al. and U.S. Pat. No. 6,383,185 to Baumgart, both of which are incorporated herein by reference.

By noninvasively monitoring the osteotomy site while simultaneously stimulating the site with ultrasound waves, distraction osteogenesis treatment times and morbidity will decrease and patient sensitive distraction vectors will result in an optimization of therapy. Therapy will be further optimized through a wireless transmitter that will allow the practitioner to remotely monitor and control the device.

An implantable sensor guided wireless distractor operates independent of patient input, decreases morbidities including scarring, nerve damage, lymphatic obstruction and muscular scarring, decreases the potential for relapse, results in decreased intraoperative blood loss as compared to long bone intramedullary distraction devices, reduces the potential for infection, eliminates the need for repeated exposure to radiation, allows for remote monitoring and operational control, incorporates mechanical safety stops and is sensitive to patient comfort and psychosocial factors. Another advantage of a sensor guided implantable distractor is that such systems may remove the need to use exogenous bone (e.g., cadaveric bone), which carries the potential for disease transmission, allergic reactions, etc.

The current art induces scarring, is prone to infection, relies heavily on patient compliance and requires bulky components that are socially unacceptable. By noninvasively monitoring the osteotomy site while simultaneously stimulating the site with ultrasound waves, distraction osteogenesis treatment times and morbidity will decrease and patient sensitive distraction vectors will result in an optimization of therapy. Therapy will be further optimized through a wireless transmitter that will allow the practitioner to remotely monitor and control the device reducing postoperative visits and radiographic exposure of the patient.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A bone distractor comprising:
a first member couplable to a first portion of a bone to be distracted during use;
a second member couplable to a second portion of a bone to be distracted at a position spaced across a separation in the bone from the attachment site of the first member during use;
an actuator coupling the first member and the second member, wherein the actuator moves the second member relative to the first member during use;
an sensor unit, wherein the sensor unit is positioned to determine physical characteristics of the callus between the first member and the second member during use, and
a controller coupled to the actuator and the sensor unit, wherein the controller determines physical characteristics of the bone based on sensor readings obtained from the sensor unit during use, and wherein the controller operates the actuator in response to the determined physical characteristics of the bone during use.

2. The distractor of claim 1, wherein the sensor unit is an ultrasound unit, wherein the controller determines physical characteristics of the bone based on ultrasound readings obtained from the ultrasound unit during use.

3. The distractor of claim 1 wherein the bone distractor is implantable.

4. The distractor of claim 1, further comprising a transmitter/receiver capable of transmitting and/or receiving control signals during use.

5. The distractor of claim 1, further comprising one or more reference elements coupled to the first member and/or the second member, wherein the controller determines physical characteristics of the bone based on sensor readings obtained from the sensor unit and readings obtained from the one or more reference elements during use.

6. The distractor of claim 1, wherein the sensor unit comprises a transmitter element and a receiver element, wherein the transmitter element is coupled to the first portion of the bone, and wherein the receiver element is coupled to the second portion of the bone.

7. The distractor of claim 6, wherein the transmitter unit sends an acoustic signal to the receiver unit along the longitudinal axis of the bone.

8. The distractor of claim 1, wherein the sensor unit comprises a transmitter element and a receiver element, wherein the transmitter element is coupled to the first portion of the bone and the second portion of the bone, and wherein the receiver element is coupled to the first portion of the bone and the second portion of the bone.

9. The distractor of claim 8, wherein the transmitter and receiver are mounted on opposing sides of an osteotomy.

10. The distractor of claim 1, wherein the controller further operates the actuator based on a predetermined data set programmed into the controller.

11. The distractor of claim 1, wherein the controller alters a distraction rate produced by the distractor in response to the determined physical characteristics of the bone during use.

12. The distractor of claim 1, wherein the sensor unit comprises an ultrasound sensor unit and wherein the controller sends one or more ultrasound pulses from the sensor unit through the callus to stimulate healing of the callus.

13. The distractor of claim 1, further comprising one or more external state sensors coupled to the controller, wherein the external state sensors measure the biological environment.

14. The distractor of claim 1, further comprising one or more internal state sensors coupled to the controller, wherein the internal state sensors measure parameters of the distractor system.

15. A method of distracting a bone comprising:
coupling a first member of a distractor to a first portion of the bone;
coupling a second member of the distractor to a second portion of the bone at a position spaced across a separation in the bone from the attachment site of the first member;
repeatedly performing:
operating a sensor unit positioned in, or proximate to, the distractor to send sensing signals to the callus;
determining the physical characteristic of the callus based on the sensing signals; and
moving the first and second member away from each other at a rate based, in part, on the determined physical characteristics of the regenerate.

16. The method of claim 15, wherein the sensor unit is an ultrasound unit, and wherein the sensing signals are ultrasound signals.

17. The method of claim 15, further comprising sending one or more ultrasound pulses through the callus to stimulate healing of the callus.

18. The method of claim 15, wherein the distraction device comprises an actuator configured to move the first and second members away from each other, and wherein operation of the actuator is controlled by a controller.

19. The method of claim 18, operation of the further comprising transmitting and/or receiving control signals to a transmitter/receiver coupled to the controller.

20. The method of claim 18, wherein the controller further operates the actuator based on a predetermined data set programmed into the controller.

21. The method of claim 15, wherein one or more reference elements are coupled to the first member and/or the second member, and wherein the physical characteristic of the callus is based on sensor readings obtained from the sensor unit and readings obtained from the one or more reference elements.

22. The method of claim 15, wherein the sensor unit comprises a transmitter element and a receiver element, wherein the transmitter element is coupled to the first portion of the bone, and wherein the receiver element is coupled to the second portion of the bone.

23. The method of claim 22, further comprising sending, from the transmitter unit, an acoustic signal to the receiver unit along the longitudinal axis of the bone.

24. The method of claim 15, wherein the sensor unit comprises a transmitter element and a receiver element, wherein the transmitter element is coupled to the first portion of the bone and the second portion of the bone, and wherein the receiver element is coupled to the first portion of the bone and the second portion of the bone.

25. The method of claim 24, wherein the transmitter and receiver are mounted on opposing sides of an osteotomy.
